(19) United States
(12) Patent Application Publication
(10) Pub. No.: US 2025/0270659 A1
(43) Pub. Date: Aug. 28, 2025

(54) METHOD FOR RAPID VERIFICATION OF ANTHOCYANIN BIOSYNTHESIS GENE FUNCTION USING ANTHURIUM ANDRAEANUM 'XUEYU'

(71) Applicant: Tropical Crops Genetic Resources Institute, CATAS, Haikou City (CN)

(72) Inventors: Zhiying LI, Haikou City (CN); Yonglin JING, Haikou City (CN); Li XU, Haikou City (CN); Cui TANG, Haikou City (CN)

(21) Appl. No.: 18/977,511

(22) Filed: Dec. 11, 2024

(30) Foreign Application Priority Data
Feb. 23, 2024 (CN) 202410202773.1

(51) Int. Cl. C12Q 1/6895; C12N 15/82; G01N 33/50

(52) U.S. Cl. CPC C12Q 1/6895; C12N 15/8205; G01N 33/5097; C12N 2503/00; C12Q 2600/13

(57) ABSTRACT

A method for rapid verification of an anthocyanin biosynthesis gene function using Anthurium andraeanum ‘Xueyu’ is provided. An engineered bacterium carrying an anthocyanin biosynthesis-related functional gene AnUFGT1 from Anthurium andraeanum is injected into a young pedicel of the Anthurium andraeanum ‘Xueyu’ once a day for 3 consecutive times using a syringe. The anthocyanin biosynthesis on a white spathe is observed to verify a function of the AnUFGT1 gene in regulating the anthocyanin biosynthesis. The method has a simple process flow, including using a non-blossomed inflorescence of the Anthurium andraeanum ‘Xueyu’ as a material, injecting a bacterial suspension into the material, conducting routine management, and then observing a phenotype after the spathe is unfolded. The method also shows a short cycle taking 15 d to 20 d during the entire process. The method provides an efficient means for rapid verification of partial functional genes.

### CROSS REFERENCE TO RELATED APPLICATION

This patent application claims the benefit and priority of Chinese Patent Application No. 202410202773.1, filed with the China National Intellectual Property Administration on Feb. 23, 2024, the disclosure of which is incorporated by reference herein in its entirety as part of the present application.

### REFERENCE TO SEQUENCE LISTING

A computer readable XML file entitled “GWP20240705279-SEQUENCE LISTING”, that was created on Sep. 6, 2024, with a file size of about 4,656 bytes, contains the sequence listing for this application, has been filed with this application, and is hereby incorporated by reference in its entirety.

### TECHNICAL FIELD

The present disclosure belongs to the technical field of agricultural biology, and in particular relates to a method for rapid verification of an anthocyanin biosynthesis gene function using Anthurium andraeanum ‘Xueyu’.

### BACKGROUND

Anthurium andraeanum Linden is a perennial herb of the genus Anthurium in the family Araceae. Such a plant has oblong heart-shaped or ovate heart-shaped leaves, heart-shaped spathes, leathery texture with waxy luster, and rich flower colors. Therefore, the Anthurium andraeanum can be used as a potted flower or cut flower, has a high ornamental value, and thus is one of the three major tropical flowers in the world.

The most unique part of Anthurium andraeanum is a heart-shaped spathe. There is a great demand for Anthurium andraeanum varieties with different flower colors in the international market. Anthurium andraeanum ‘Xueyu’ (Plant Variety Rights NO: CAN20090270.0) is a white bud mutant of Anthurium andraeanum ‘Alabama’, and metabolome analysis shows almost no anthocyanin detected. Key functional genes in the anthocyanin biosynthesis pathway play an important role in the color development, and a flavonoid 3-O-glycosyltransferase (UFGT) gene can promote anthocyanin glycosidation to form stable anthocyanins that can accumulate in vacuoles.

In red spathes, anthocyanins are mainly cyanins with a small amount of pelargonidin (Iwata et al., 1985). They all come from a flavonoid biosynthesis pathway. First, chalcone synthase (CHS) catalyzes the production of naringenin chalcone from 4-formyl-coenzyme A. The enzymes subsequently implicated include CHI, F3H, DFR, ANS, UF3GT, and RT/GT/MT. The intermediates formed include naringin/diene-diol, dihydroflavonol, leucoanthocyanin, and anthocyanin (Vern, 2002). In the anthocyanin biosynthesis pathway, a UDP-glucose: flavonoid 3-O-glucosyltransferase (UF3GT) gene is the last structural gene that catalyzes anthocyanin 3-O-glucosyltransferase. The structural gene converts unstable anthocyanins into stable anthocyanins. In most plants, anthocyanin accumulation is associated with the expression of UF3GT gene.

Genetic modification is the most direct method to verify gene function, but there is a prerequisite to establish a genetic transformation system and then observe a phenotype after plant regeneration to determine the gene function, which is difficult, time-consuming, and labor-intensive with a long cycle. LI Xiuxiu (2019) used the stem segments of Anthurium andraeanum as explants, and induced callus and adventitious buds after Agrobacterium infection. SHENG Hui et al. (2017) used the leaves of Anthurium andraeanum as explants, and established an efficient regeneration system through callus induction, adventitious bud differentiation, and rooting culture. KONG Zhouyang (2017) used the leaves of Anthurium andraeanum as explants, and obtained adventitious buds after callus induction. Dao Thanh Luong (2012) also used the leaves of Anthurium andraeanum tissue culture seedlings as explants, and then obtained transformants through callus and adventitious bud differentiation after Agrobacterium infection. LIU Huichun (2012) used the leaves of Anthurium andraeanum ‘Alabama’ as explants, and transformed a green fluorescent protein gene GFP and a fatty acid desaturase gene FAb 3 into Anthurium andraeanum through Agrobacterium-mediated transformation, such that a transgenic Anthurium andraeanum plant was obtained through G418 and kanamycin screening. The transformation method used to identify the function of the spathe color regulatory gene has high operational requirements, great difficulty, complex technical links, and a long cycle taking 1 to 2 years from transplanting to flowering.

### SUMMARY

In order to solve the above technical problems, the present disclosure provides a method for rapid verification of an anthocyanin biosynthesis gene function using Anthurium andraeanum ‘Xueyu’. In the present disclosure, a non-blossomed inflorescence of the Anthurium andraeanum ‘Xueyu’ is used as a material, and a bacterial suspension of a target gene is directly injected into a pedicel using a syringe. After 15 d to 20 d, phenotypic changes can be observed after the spathe is fully unfolded. The method adopts direct injection, with a process that is simple and easy to master, and does not require infection, co-cultivation, screening culture, proliferation, rooting, and transplanting in genetic transformation using explants such as Anthurium andraeanum leaves and stem segments. The method requires a short cycle, taking only 15 d to 20 d from the end of injection to the observation of phenotype. The method also has a simple process flow with high efficiency, which can quickly verify the function of genes.

To achieve the above objective, the present disclosure adopts the following technical solutions:

The present disclosure provides a method for rapid verification of an anthocyanin biosynthesis gene function using Anthurium andraeanum ‘Xueyu’, including the following steps: introducing an Agrobacterium strain with an AntIFGT1 gene of Anthurium andraeanum into a pedicel of an unfolded young spathe of the Anthurium andraeanum ‘Xueyu’ using a syringe, and conducting phenotypic observation immediately after the unfolded young spathe unfolds; where

the introducing specifically includes the following steps: piercing the pedicel at a position of 1 cm to 3 cm below the unfolded spathe to form a first injection point by diagonally piercing 2 mm to 3 mm downward, gently and slowly pushing the syringe by 1 μL to 3 μL to allow first injection, and applying vaseline around an obtained needle hole while pulling out the syringe on a first day; conducting second injection at a position 0.4 cm to 0.6 cm upward from the first injection point on a same pedicel to form a second injection point on a second day; and conducting third injection at a position 0.4 cm to 0.6 cm upward from the second injection point on the same pedicel to obtain an injected young spathe.

Preferably, the method specifically includes the following steps:
(1) selecting an Anthurium andraeanum ‘Xueyu’ plant with the unfolded young spathe;
(2) collecting bacterial cells with an expression vector of the AnUFGT1 gene, subjecting the bacterial cells to resuspension until an OD₆₀₀ value reaches 0.8 to 1.0, and then collecting 10 μL to 20 μL of a resulting bacterial suspension using a sterile insulin syringe;
(3) piercing the pedicel at the position of 1 cm to 3 cm below the unfolded spathe to form the first injection point by diagonally piercing 2 mm to 3 mm downward, gently and slowly pushing the syringe by 1 μL to 3 μL to allow the first injection, and applying the vaseline around the obtained needle hole while pulling out the syringe on the first day; conducting the second injection at the position 0.4 cm to 0.6 cm upward from the first injection point on the same pedicel to form the second injection point on the second day; and conducting the third injection at the position 0.4 cm to 0.6 cm upward from the second injection point on the same pedicel to obtain the injected young spathe;
(4) placing a resulting injected Anthurium andraeanum ‘Xueyu’ plant back to the greenhouse to allow normal management with insect control; and
(5) cutting an inflorescence promptly from a base 14 d to 15 d later when the injected young spathe is fully unfolded before flowering, inserting the inflorescence immediately into clean water and bringing to a laboratory to allow the phenotypic observation and photographic recording using a stereo microscope.

Preferably, the Anthurium andraeanum ‘Xueyu’ plant selected in step (1) belongs to a white variety lacking the anthocyanin (CAN20090270.0), has a seedling age of 2 to 3 years, blooms normally under room-temperature cultivation, and then is stored in National Gene Bank of Tropical Crops.

Preferably, primers for cloning the AnUFGT1 gene in step (2) have sequences shown in SEQ ID NO: 1 to SEQ ID NO: 4.

Preferably, the expression vector in step (2) is constructed with a vector PBI121, restriction endonucleases Kpn I and Xba I, an Escherichia coli strain DH5α, and an Agrobacterium strain EHA105.

Preferably, the resuspension in step (2) is conducted using a solution of 4% to 6% of sucrose+0.01% to 0.03% of Silwet™ L-77 silicon surfactant.

More preferably, the resuspension is conducted using a solution of 5% of the sucrose+0.02% of the Silwet™ M L-77 silicon surfactant.

Preferably, step (3) specifically includes: piercing the pedicel at the position of 2 cm below the unfolded spathe to form the first injection point by diagonally piercing 2 mm to 3 mm downward, gently and slowly pushing the syringe by 2 μL to allow the first injection, and applying the vaseline around the obtained needle hole while pulling out the syringe on the first day; conducting the second injection at the position 0.5 cm upward from the first injection point on the same pedicel to form the second injection point on the second day; and conducting the third injection at the position 0.5 cm upward from the second injection point on the same pedicel to obtain the injected young spathe.

Compared with the prior art, the present disclosure has the following beneficial effects:

In the present disclosure, the method has a simple process flow with high efficiency, a non-blossomed inflorescence of the Anthurium andraeanum ‘Xueyu’ is used as a material, and a bacterial suspension of a target gene is directly injected into a pedicel using a syringe. After 15 d to 20, phenotypic changes can be observed after the spathe is fully unfolded. The method adopts direct injection, with a process that is simple and easy to master, and does not require infection, co-cultivation, screening culture, proliferation, rooting, and transplanting in genetic transformation using explants such as Anthurium andraeanum leaves and stem segments. The method requires a short cycle, taking only 15 d to 20 from the end of injection to the observation of phenotype, thus quickly verifying the function of genes.

### BRIEF DESCRIPTION OF THE DRAWINGS

FIGS. 1A-1C show appearances of the Anthurium andraeanum ‘Xueyu’ plant in Example 1 of the present disclosure, where FIG. 1A is a flowering plant of the Anthurium andraeanum ‘Xueyu’; FIG. 1B is a comparison of the spathes of Anthurium andraeanum ‘Xueyu’ and its wild type ‘Alabama’; and FIG. 1C is an appearance of the Anthurium andraeanum ‘Xueyu’ plant for injection; and

FIGS. 2A-2D show effect analysis after the injection in Example 1 of the present disclosure, where FIG. 2A is the young spathe of Anthurium andraeanum ‘Xueyu’ for injection; FIG. 2B is the unfolded spathe after injection (without the spike); FIG. 2C is the red spot on the spathe; and FIG. 2D is the detection results of the composition and content of the red spot, indicating that it is anthocyanin.

### DETAILED DESCRIPTION OF THE EMBODIMENTS

The following examples are intended to illustrate the present disclosure, but not to limit the scope of the present disclosure. Modifications or substitutions made to methods, steps or conditions of the present disclosure without departing from the spirit and essence of the present disclosure fall within the scope of the present disclosure. The reagents, kits, and instruments used in the following examples are all commercially available, and the methods used in the examples are consistent with conventional methods unless otherwise specified.

The technical solutions of the present disclosure will be further described in detail below with reference to examples.

### Example 1

This example provided a method for rapid verification of an anthocyanin biosynthesis gene function using Anthurium andraeanum ‘Xueyu’, including the test materials, functional genes, preparation of Agrobacterium, and injection process, specifically:

### 1. Experimental Materials

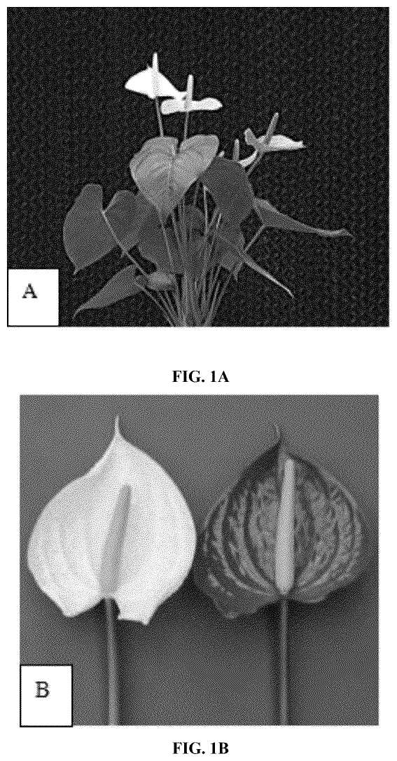

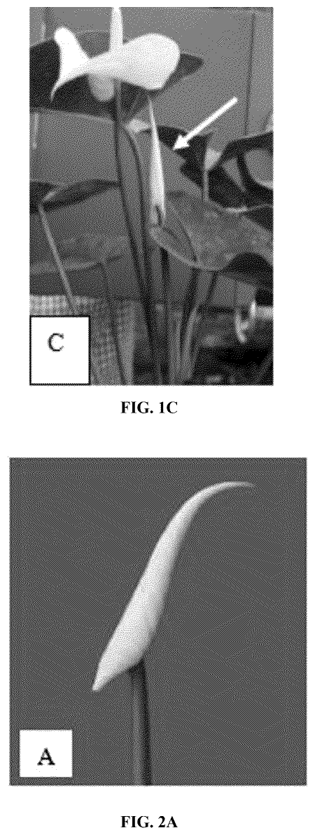

An anthocyanin-deficient mutant variety, Anthurium andraeanum ‘Xueyu’, was selected with a seedling age of 2 to 3 years and then cultivated at room temperature. The unfolded spathes were selected during the flowering period (FIGS. 1A-1C).

### 2. Cloning of Functional Gene and Construction of Expression Vector

AnUFGT1, a key gene for anthocyanin biosynthesis was obtained from Anthurium andraeanum. A cDNA was amplified by PCR from total RNA transcribed from leaves of Anthurium andraeanum ‘Alabama’ with the following primers: AnUFGT1-F (5′-CATCGCCTG CTCCGCC-3′) (SEQ ID NO: 1) and AnUFGT1-R (5′-CAAACTCGGACAAACTACGCC-3′) (SEQ ID NO: 2), and the PCR program included: 94° C. for 5 min; 35 cycles of the following program: 94° C. for 30 s, 60° C. for 30 s, 72° C. for 2 min; and 72° C. for 10 min. An amplified product was sequenced and analyzed, and the results were uploaded to an international data platform figshare with the name: The cDNA sequence of An UFGT 1.

An amplified product was used as a template and PCR amplification was conducted using primers AnUFGT1-FK (5′-CAAACTCGGACA AACTACGCC-3′) (SEQ ID NO: 3) and AnUFGT1-RX (5′-GCTCTAGACAACTCGGACAAAACTACGCC-3′) (SEQ ID NO: 4), and the PCR program included: 98° C. for 4 min; 35 cycles of the following program: 98° C. for 10 s, 60.5° C. for 5 s, 72° C. for 1.5 min; and 72° C. for 10 min. After PCR products were detected by 1.2% agarose gel electrophoresis, a target fragment was recovered and ligated into a cloning vector pEASY®-Blunt Cloning Vector. A plasmid AnUFGT1-Blunt was extracted using the Plasmid Mini Kit from OMEGA, and a pBI121 plasmid was prepared at the same time. The plasmids AnUFGT1-T1 and pBI121 were double-digested with restriction endonucleases Kpn I and Xba I, respectively, and electrophoresed on 1.2% agarose gel. The separated target fragments were recovered, ligated using a T4 DNA ligase from TransGen, transformed into Escherichia coli DH5α, and positive clones were identified. A plasmid of the positive clones was extracted, transformed into an Agrobacterium tumefaciens strain EHA105, and positive bacteria were identified, added into an equal volume of 50% glycerol, and then stored in a −80° C. refrigerator for later use.

### 3. Genetic Transformation

10 μL of Agrobacterium bacterial solution containing positive clones was inoculated into 1 mL of LB broth (containing 50 μg/mL kanamycin and 100 μg/mL rifampicin), and cultured at 28° C., 220 rpm for about 30 h; 500 μL of an activated bacterial solution was inoculated into 200 mL of LB broth (containing 50 μg/mL kanamycin and 100 μg/mL rifampicin), and cultured at 28° C., 220 rpm overnight; the OD₆₀₀ value was determined. When the OD₆₀₀ value was 0.8 to 1.2, the bacterial cells were collected by centrifugation at 4,000×g for 10 min at 4° C., and resuspended in a solution of 5% sucrose+0.02% Silwet™ L-77 silicon surfactant until the OD₆₀₀ value reached 0.8 to 1.0.

(10-20) μL of the bacterial solution was selected with a sterile insulin syringe, the pedicel was pierced at a position of 2 cm below the unfolded spathe to form a first injection point by diagonally piercing 2 mm to 3 mm downward, the syringe was gently and slowly pushed by 2 μL to allow first injection, and vaseline was applied around an obtained needle hole while the syringe was pulled out on a first day. Second injection was conducted at a position 0.5 cm upward from the first injection point on a same pedicel to form a second injection point on a second day; and third injection was conducted at a position 0.5 cm upward from the second injection point on the same pedicel to obtain an injected young spathe.

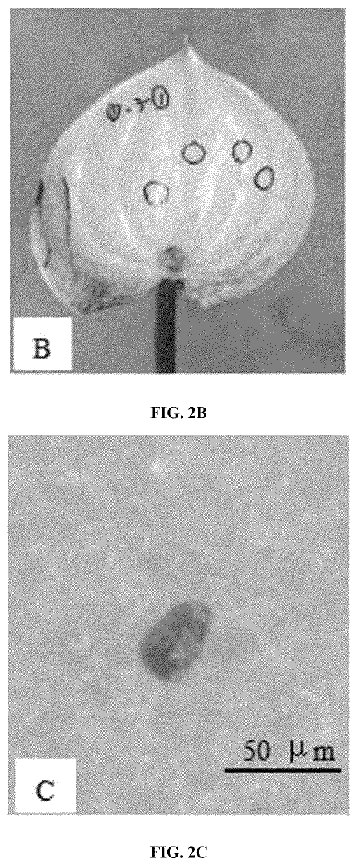

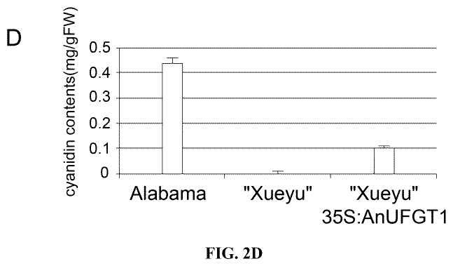

A resulting injected Anthurium andraeanum ‘Xueyu’ plant was placed back to the greenhouse to allow normal management. An inflorescence promptly was cut from a base 14 d to 15 d later when the injected young spathe was fully unfolded, and the inflorescence was inserted immediately into clean water and brought to a laboratory to allow the phenotypic observation and photographic recording using a stereo microscope. The observation and recording results were shown in FIGS. 2A-2D.

4. Determination of anthocyanin Content

Standard solutions of 28.0 μg/mL, 11.2 μg/mL, 5.60 μg/mL, 2.80 μg/mL, and 0 μg/mL were prepared using standard samples of cyanidin, pelargonidin, and delphinidin, respectively, while 0.1% hydrochloric acid-methanol solution was used as a blank control. The absorbance was detected at a maximum absorption wavelength of 530 nm using a visible-UV spectrophotometer. The detection was repeated in parallel for 3 times, and a standard curve was drawn.

Fresh spathe samples were ground into a powder in liquid nitrogen, extracted with 0.1% hydrochloric acid-methanol solution, centrifuged, a resulting supernatant was mixed with chloroform, centrifuged, and a resulting supernatant was diluted with an equal volume of the 0.1% hydrochloric acid-methanol solution. The absorbance at 530 nm was detected by UV spectrophotometer and substituted into a standard curve equation. Based on the results, whether the red spots were anthocyanin was determined and the anthocyanin content was calculated.

3 batches were injected, with 10 flowers injected in each batch. Statistical results showed that 3 to 4 flowers had red spots each time, and there were 10 to 17 of the red spots per batch.

The above examples are only intended to describe the preferred implementations of the present disclosure, but not to limit the scope of the present disclosure. Various alterations and improvements made by those of ordinary skill in the art based on the technical solution of the present disclosure without departing from the design spirit of the present disclosure shall fall within the scope of the appended claims of the present disclosure.

What is claimed is:

1. A kit for rapid verification of an anthocyanin biosynthesis gene function, comprising Anthurium andraeanum ‘Xueyu’, a syringe, primers and a suspension solution.

2. The kit according to claim 1, wherein the Anthurium andraeanum ‘Xueyu’ is a white variety lacking anthocyanin with a Plant Variety Rights NO. of CAN20090270.0, has a seedling age of 2 to 3 years, blooms normally under room-temperature cultivation, and is stored in National Gene Bank of Tropical Crops.

3. The kit according to claim 2, wherein the primers have sequences shown in SEQ ID NO: 1 to SEQ ID NO: 4.

4. The kit according to claim 3, wherein the suspension solution is a solution of 4% to 6% of a sucrose+0.01% to 0.03% of a silicon surfactant containing polycyclic ether-modified polydimethylsiloxane and polyoxyethylene polyoxypropylene copolymer as active ingredients.

5. A method for using the kit according to claim 1, comprising
introducing an Agrobacterium strain with an AnUFGT1 gene of Anthurium andraeanum into a pedicel of an unfolded young spathe of the Anthurium andraeanum ‘Xueyu’ using a syringe, and conducting phenotypic observation immediately after the unfolded young spathe unfolds; wherein
the introducing specifically comprises the following steps: piercing the pedicel at a position of 1 cm to 3 cm below the unfolded spathe to form a first injection point by diagonally piercing 2 mm to 3 mm downward, gently and slowly pushing the syringe by 1 μL to 3 μL to allow first injection, and applying vaseline around an obtained needle hole while pulling out the syringe on a first day; conducting second injection at a position 0.4 cm to 0.6 cm upward from the first injection point on a same pedicel to form a second injection point on a second day; and conducting third injection at a position 0.4 cm to 0.6 cm upward from the second injection point on the same pedicel to obtain an injected young spathe.

6. The method according to claim 5, specifically comprising the following steps:
(1) selecting an Anthurium andraeanum ‘Xueyu’ plant with the unfolded young spathe;
(2) collecting bacterial cells with an expression vector of the AnUFGT1 gene, subjecting the bacterial cells to resuspension until an OD₆₀₀ value reaches 0.8 to 1.0, and then collecting 10 μL to 20 μL of a resulting bacterial suspension using a sterile insulin syringe;
(3) piercing the pedicel at the position of 1 cm to 3 cm below the unfolded spathe to form the first injection point by diagonally piercing 2 mm to 3 mm downward, gently and slowly pushing the syringe by 1 μL to 3 μL to allow the first injection, and applying the vaseline around the obtained needle hole while pulling out the syringe on the first day; conducting the second injection at the position 0.4 cm to 0.6 cm upward from the first injection point on the same pedicel to form the second injection point on the second day; and conducting the third injection at the position 0.4 cm to 0.6 cm upward from the second injection point on the same pedicel to obtain the injected young spathe;
(4) placing a resulting injected Anthurium andraeamum ‘Xueyu’ plant back to the greenhouse to allow normal management with insect control; and
(5) cutting an inflorescence promptly from a base 14 d to 15 d later when the injected young spathe is fully unfolded but before flowering, and inserting the inflorescence immediately into clean water and bringing to a laboratory to allow the phenotypic observation and photographic recording using a stereo microscope.

7. The method according to claim 6, wherein the Anthurium andraeanum ‘Xueyu’ is a white variety lacking anthocyanin with a Plant Variety Rights NO. of CAN20090270.0, has a seedling age of 2 to 3 years, blooms normally under room-temperature cultivation, and is stored in National Gene Bank of Tropical Crops.

8. The method according to claim 7, wherein the primers for cloning the AnUFGT1 gene in step (2) have sequences shown in SEQ ID NO: 1 to SEQ ID NO: 4.

9. The method according to claim 8, wherein the expression vector in step (2) is constructed with a vector PBI121, restriction endonucleases Kpn I and Xba I, an Escherichia coli strain DH5α, and an Agrobacterium strain EHA105.

10. The method according to claim 8, wherein the resuspension in step (2) is conducted using a solution of 4% to 6% of a sucrose+0.01% to 0.03% of a silicon surfactant containing polycyclic ether-modified polydimethylsiloxane and polyoxyethylene polyoxypropylene copolymer as active ingredients.

11. The method according to claim 10, wherein step (3) specifically comprises: piercing the pedicel at the position of 2 cm below the unfolded spathe to form the first injection point by diagonally piercing 2 mm to 3 mm downward, gently and slowly pushing the syringe by 2 μL to allow the first injection, and applying the vaseline around the obtained needle hole while pulling out the syringe on the first day; conducting the second injection at the position 0.5 cm upward from the first injection point on the same pedicel to form the second injection point on the second day; and conducting the third injection at the position 0.5 cm upward from the second injection point on the same pedicel to obtain the injected young spathe.


SEQUENCE LISTING (4 sequences)

SEQ ID NO: 1 - DNA, 16 bp, synthetic construct
  source 1..16: mol_type = other DNA
catcgcctgctccgcc

SEQ ID NO: 2 - DNA, 21 bp, synthetic construct
  source 1..21: mol_type = other DNA
caaactcggacaaactacgcc

SEQ ID NO: 3 - DNA, 21 bp, synthetic construct
  source 1..21: mol_type = other DNA
caaactcggacaaactacgcc

SEQ ID NO: 4 - DNA, 28 bp, synthetic construct
  source 1..28: mol_type = other DNA
gctctagacaactcggacaaactacgcc